SEQUENCE LISTING (17 sequences)

SEQ ID NO: 1 - protein, 701 aa, Thermus phage phiFa
MNKTELVKKLIEEKTAELAVRPVYTGRIDEEGQWTPTSLHERYQDKVLVVPAPEEGEPVQTIGEYEFVGGNSNRIVYIHRSIIKQWLEVAGGDSHWGAAVVLRPGKLASHNAVFSWEGYQLEESDEGLLATMDERDWKVLLEAYDLDPEVRLVQVTVFDLEQGVSAKGTLRKVLPGERPVVFPNSWKKFGNLRTQFMSILTTDVHYNPRASVNLQELTYYVQEAPAQWVFDALDEEIERIINLKTSPWLTTGYLTRLGFSPAWNSTLTALKESLCGELKKLLRRIKLSGKYGLRAKSTSSHLLRDGELLLPAYAKKHVKVGDFVTVSRNPALPSQGWAKYRVTGFIDGNMVVFPQGDYAWSAILGGDHDGDDAVVFYRAPVEGYELPDDTLDLQAIKPVARKLDANTVEARIQRWQNEVSVNIGQFDLAARRLLAANKLHREEAKLLSVAIQTAISLKKRVARLEDQSWWPRVAELLEESKKLAGTTWVDMIRDGREPVGAPDWVLTIYQKVHEAIKKVQKLEPRLWLTEAKVREFAKGATVPASCEALIQYREKLLKAKAIAVQNEDGDTVARLNKELKEFIHVEAPKHLLGIDEEDWKQFSRWAIGNTHISEWVFWCHPAILEELYDRFGKQTIKAIALGGTPNLAESEVIELEDQELIDRVFEVEGVKYALLDETGYLKAGRLKVTKVRKSVVEFEYA

SEQ ID NO: 2 - DNA, 2934 bp, synthetic construct
  source 1..2934: mol_type = other DNA
atgaacaaaaccgagttagttaagaaactcatagaggaaaaaactgcagaactcgctgtacgtcctgtttataccgggaggatcgatgaggaggggcagtggactcctacttcgctgcacgaacgttatcaggataaggttctggtagtacctgcacctgaagaaggggagccggttcagaccatcggtgagtatgagtttgttggggggaactcgaacaggatcgtttatatccatcgttcgatcatcaaacaatggctggaggttgcagggggtgacagccactggggggcagcagtagtactgcgtccgggtaaactcgctagccataacgcagttttttcgtgggagggttatcagttagaggaatcggatgagggtctgctggcaactatggacgaacgtgattggaaagtactgctggaggcttatgatctggatcctgaagtacgtctcgttcaggttactgttttcgatctggagcagggtgttagcgcaaaaggtactctgcgtaaagttctccctggggagcgtcctgtagtttttcctaactcgtggaaaaaatttggtaacctccgtactcaatttatgagcatcctgaccactgacgttcactataacccccgtgctagcgttaacctacaagagctcacctattatgttcaggaggctcccgctcagtgggtttttgacgctctggatgaagagatcgaacgtatcataaacctgaaaaccagcccttggctgactactggttatctgacccgtctcggtttcagccctgcttggaactcgaccctcaccgctctcaaggaaagcctgtgcggtatgaacaaaaccgagttagttaagaaactcatagaggaaaaaactgcagaactcgctgtacgtcctgtttataccgggaggatcgatgaggaggggcagtggactcctacttcgctgcacgaacgttatcaggataaggttctggtagtacctgcacctgaagaaggggagccggttcagaccatcggtgagtatgagtttgttggggggaactcgaacaggatcgtttatatccatcgttcgatcatcaaacaatggctggaggttgcagggggtgacagccactggggggcagcagtagtactgcgtccgggtaaactcgctagccataacgcagttttttcgtgggagggttatcagttagaggaatcggatgagggtctgctggcaactatggacgaacgtgattggaaagtactgctggaggcttatgatctggatcctgaagtacgtctcgttcaggttactgttttcgatctggagcagggtgttagcgcaaaaggtactctgcgtaaagttctccctggggagcgtcctgtagtttttcctaactcgtggaaaaaatttggtaacctccgtactcaatttatgagcatcctgaccactgacgttcactataacccccgtgctagcgttaacctacaagagctcacctattatgttcaggaggctcccgctcagtgggtttttgacgctctggatgaagagatcgaacgtatcataaacctgaaaaccagcccttggctgactactggttatctgacccgtctcggtttcagccctgcttggaactcgaccctcaccgctctcaaggaaagcctgtgcggtgaactgaagaaactactgcgtcgtataaaactgtcgggtaaatatggtctgcgtgcaaaatcgacttcgagccacctcctgcgtgatggtgaattactgttacctgcatacgcaaaaaaacacgttaaggttggtgatttcgttactgtttcgcgtaaccctgcactaccttcgcaggggtgggcaaaatatcgtgtaactgggtttatagatggtaacatggtagtttttcctcagggggattatgcatggagcgctatcctgggtggtgatcatgatggtgacgacgcagttgtattttatcgtgctccggttgaaggttatgaactcccggacgacactctggatctgcaggcaataaaacccgtagcacgtaaactcgacgcaaacaccgtagaggcaaggatacaacgttggcaaaacgaggtaagcgttaacatcggtcagttcgatctggctgctaggcgtctcctagcagcaaacaagctgcatcgtgaggaggcaaaactactgtcggtagcaatacagactgcaatcagcctcaaaaaacgtgtagctcgtctagaagatcagagctggtggccgcgtgtagctgagctgctcgaagagagcaaaaaactcgctggtaccacttgggttgatatgatacgtgatggtcgtgagccggtaggggcacccgactgggttctcaccatctatcagaaagttcatgaagcaatcaagaaagttcagaagctggaaccccgtctgtggctcactgaagcaaaagtacgtgaattcgcaaagggggcaactgtacctgcaagctgcgaggctctaatccagtatcgtgaaaaactcctgaaagctaaagcaatcgcagttcagaacgaggatggtgatactgttgcacgtttaaacaaagaactgaaggagtttatccacgtagaggcaccgaaacatctgctggggatagatgaagaagattggaaacagtttagcaggtgggcaatcgggaacacccatatcagcgaatgggttttttggtgccaccccgctatcctagaagaactgtatgatcgtttcgggaaacaaaccatcaaagctatcgcactgggtgggactccgaacctagcagaaagcgaggtaatcgagctggaagatcaggaactaatagatcgtgtatttgaagttgaaggtgtaaaatatgctctgttagatgaaaccggttatctcaaggcagggcgtctgaaagttactaaggttcgtaaatcggttgtagaatttgaatatgcttga

SEQ ID NO: 3 - DNA, 468 bp, synthetic construct
  source 1..468: mol_type = other DNA
aattctaatacgactcactatagtatccggtgggccagccagcgtaccgtcctcgaggctgtggattgcgcggcttcccgcccgcagccgcgcatctagtcctcccgccaccaccacctctgcaggaagaggtgggcgaagagacgaagagaggtgaaagagagaagaggcgaaggccgggtgtcccgcgcccggctcgccttacggtgccacgccaccccgctcgcgcaaggctccgcgcggagcgcggccccttcgggcttacggccttgcgctcgcggggaccccggcgtggcagggctccgccccgagccgggcgcgtagaggacacccggctcaaggaggtcaaagatgaacaagaccgaacttgtcaaaaagttgattgaagagaagacggcggagttggctgttaggccggtgtacaccggtcgaatcgacgaagaaggtcagtggactccgacttctctg

SEQ ID NO: 4 - DNA, 750 bp, synthetic construct
  source 1..750: mol_type = other DNA
ctgtggacgctgacttcatcgacgtactggaagatgtcaaaaattgaggcgtttgtggtataattagggctgtagcaagcaggtacactcggcggcaagtctggagactggacttgccgcccctgcttttggagcgccgggtggtttgggcgcggctttatgtttcgcccatggggggagccacccggctttatcctcccccgactggaggctagatgcagcttcgcaagattgcgccgaacgccaacttcttccgcctccttgaggagaacttcagcgcggaactggaagacatggacgctctgcccatctaggaggttggatgaacatccgagatgcccccaagctgttcctcggagctttcgggctttcgaccccagctacgtggctgcggtggagagcatcgcgaagcggaagctctgagaggtgaagcatggcgaggttcactggatttagcaaggtcttctcggtcggcaagacgctggagaaccaccgcatcaagttcttcagctacgctaccgtctagaccgcgtaagggtggggagggggcagtctctccccacccacctgttacctggaggtgaacgtgcgagtgcgtttgcgtaaggcggtgtacgctagcggcaaactccatgcggcggggagtgttcaaggtgatttctggccttgtgttcttccgcgtgtgaggtgagagatggcgatttttgagctgaacgctggttccgaccttcgcttccccaccaggaagga

SEQ ID NO: 5 - DNA, 19 bp, synthetic construct
  source 1..19: mol_type = other DNA
agctgatgagtacgaactt

SEQ ID NO: 6 - DNA, 22 bp, synthetic construct
  source 1..22: mol_type = other DNA
ttcagatttttaacacgagagt

SEQ ID NO: 7 - DNA, 44 bp, synthetic construct
  source 1..44: mol_type = other DNA
accacgaaagcaagaaaaagaagtattcgtttcggaagagacag

SEQ ID NO: 8 - DNA, 44 bp, synthetic construct
  source 1..44: mol_type = other DNA
ttgctagttacactagccatccttaggttttacaagactcacgt

SEQ ID NO: 9 - DNA, 21 bp, synthetic construct
  source 1..21: mol_type = other DNA
ctgcgcttcgattgtgtgcgt

SEQ ID NO: 10 - DNA, 19 bp, synthetic construct
  source 1..19: mol_type = other DNA
agctgatgagtacgaactt

SEQ ID NO: 11 - RNA, 46 nt, synthetic construct
  source 1..46: mol_type = other RNA
attgcgacacaatattgcagcagtacgcacacaatcgaagcgcaga

SEQ ID NO: 12 - DNA, 50 bp, synthetic construct
  source 1..50: mol_type = other DNA
  misc_feature 1: note = Cy3-labelled
  misc_feature 1..50: note = Single stranded DNA
cgatggccgtagataaggcgcccggggacgaccacgtcaaggcgcagttc

SEQ ID NO: 13 - DNA, 10 bp, synthetic construct
  source 1..10: mol_type = other DNA
  misc_feature 1..10: note = Single stranded DNA
taggggcggm

SEQ ID NO: 14 - DNA, 12 bp, synthetic construct
  source 1..12: mol_type = other DNA
  misc_feature 1..12: note = Single stranded DNA
taggggcggmta

SEQ ID NO: 15 - DNA, 12 bp, synthetic construct
  source 1..12: mol_type = other DNA
  misc_feature 1..12: note = Single stranded DNA
caggggcggcaa

SEQ ID NO: 16 - DNA, 90 bp, synthetic construct
  source 1..90: mol_type = other DNA
  misc_feature 1..90: note = Single stranded DNA
gcgctccaaaagcaggggcggcaagtccagtctccagacttgccgccgagtgtacctgcttgctacagccctaattataccacaaacgcc

SEQ ID NO: 17 - DNA, 90 bp, synthetic construct
  source 1..90: mol_type = other DNA
  misc_feature 1..90: note = Single stranded DNA
ggcgtttgtggtataattagggctgtagcaagcaggtacactcggcggcaagtctggagactggacttgccgcccctgcttttggagcgc

(19) United States
(12) Patent Application Publication
(10) Pub. No.: US 2025/0101497 A1
(43) Pub. Date: Mar. 27, 2025

(54) Thermostable RNA Polymerase

(71) Applicant: Wageningen Universiteit, Wageningen (NL)

(72) Inventors: Aiko Jurre Steens, Wageningen (NL); Raymond Hubert Josèphe Staals, Ede (NL); John Van Der Oost, Renkum (NL)

(21) Appl. No.: 18/729,729

(22) PCT Filed: Jan. 20, 2023
(86) PCT No.: PCT/NL2023/050024

§ 371 Date: Jul. 17, 2024

(30) Foreign Application Priority Data
Jan. 20, 2022 (EP) 22152542.1

(51) Int. Cl. C12Q 1/6816; C12N 9/22; C12N 15/11; C12Q 1/6844

(52) U.S. Cl. CPC C12Q 1/6816; C12N 9/22; C12N 15/111; C12Q 1/6844; C12N 2310/20

(57) ABSTRACT

The invention relates to a protein having at least 50% sequence identity with SEQ ID NO:1, and its use in in vitro transcription methods. The invention further relates to a nucleic acid molecule encoding the protein and to a host cell expressing the protein.

### FIELD

The invention is directed to a novel thermostable RNA polymerase, and its use for in vitro transcription and in diagnostic methods.

### 1 INTRODUCTION

An in vitro transcription (IVT) reaction normally requires a template nucleic acid molecule comprising a promoter, ribonucleotide triphosphates, a buffer system that includes DTT and magnesium ions, and an appropriate ribonucleic acid (RNA) polymerase. Said RNA polymerase is often selected from a single subunit bacteriophage-derived RNA polymerase such as SP6, T3 and T7. Transcription is initiated by binding of an RNA polymerase to its promoter sequence, followed by feeding of the template strand into the active site, the generation of a RNA transcript, and termination of the transcription reaction.

Besides its use for IVT, an RNA polymerase is often used in nucleic acid amplification methods, especially for diagnostic purposes. For example, Nucleic Acid Sequence Based Amplification (NASBA; U.S. Pat. No. 5,654,142A) and Transcription Mediated Amplification” (TMA; WO1991001384A1) involve alternating cycli in which an RNA polymerase and a reverse transcriptase are consecutively producing each other's template under isothermal conditions. Following amplification, hybridization of a complementary oligonucleotide probe may allow detection via a detectable label, as is employed, for example, in quantitative PCR, also termed quantitative real-time PCR. In addition, certain clustered regularly interspaced short palindromic repeats (CRISPR)-CRISPR-associated (Cas) protein based nucleic acid detection systems make use of an RNA-intermediate (Steens et al., 2021. Nature Comm 12:1-12).

It would be advantageous if the reaction temperature could be increased to improve the reaction rate. For example, if the reaction temperature of an isothermal amplification is increased, it may be possible to amplify RNA having a secondary structure. However, despite the identification of RNA polymerase variants displaying higher thermostability than wildtype bacteriophage-derived RNA polymerases (Boulain et al., 2013. Protein Eng Des Sel. 26(11): 725-734), no RNA polymerase is commercially available today that remains active after more than 5 minutes incubation at 65° C.

There is thus is a need for an RNA polymerase with improved stability and activity at higher reaction temperatures.

### 2 BRIEF DESCRIPTION OF THE INVENTION

The invention provides a method comprising incubating a template nucleic acid molecule with a protein having at least 50% sequence identity with SEQ ID NO:1 in the presence of ribonucleic acid nucleotides (rNTPs) and a suitable buffer; and transcribing at least part of said template nucleic acid molecule into a RNA molecule by performing a transcription reaction at a temperature between 30° C. and 80° C. Said template nucleic acid molecule preferably is a deoxyribonucleic acid (DNA) template molecule, either a single stranded or a double stranded DNA template molecule. Said buffer preferably comprises 25-200 mM of NaCl. Said transcription reaction preferably is performed at a temperature between 45° C.-75° C. The template nucleic acid molecule may have been generated by any means, including by a pre-amplification reaction.

The transcribed RNA molecule may further be incubated with a clustered regularly interspaced short palindromic repeats (CRISPR)-CRISPR-associated (Cas) protein based nucleic acid detection system. Said CRISPR-Cas nucleic acid detection system preferably comprises a) an effector complex comprising a Type III Cas protein and at least one CRISPR RNA (crRNA) that binds a target RNA molecule; b) means for directly or indirectly determining a level of cyclic oligoadenylate (cOA). In a preferred method of the invention, the pre-amplification reaction, transcription reaction and incubation with a nucleic acid detection system are all performed in a one pot reaction.

The invention further provides a protein having at least 50% sequence identity with SEQ ID NO:1. Said protein preferably comprises a suitable tag.

The invention further provides a nucleic acid molecule encoding a protein of the invention. Said nucleic acid molecule preferably is codon optimized for expression of said protein in a suitable host cell.

The invention further provides a host cell expressing a protein of the invention.

The invention further provides a use of a protein of the invention for performing a transcription reaction to transcribe at least part of a template nucleic acid molecule into a RNA molecule. Said transcription reaction preferably is performed at a temperature between 30° C. and 80° C., preferably at a temperature between 45° C.-75° C.

### 3 FIGURE LEGENDS

FIG. 1. PhiFa_44 substrate specificity for IVT. Marker used is NEB low range ssRNA (NEB #N0364S).

FIG. 2. Analysis of phiPa_44 IVT performance under various NaCl concentrations and temperatures. A: Template only, no DNase. B: Template only, with DNase. C: PhiFa only, with DNase.

FIG. 3. SARS-COV-2 one pot reaction.

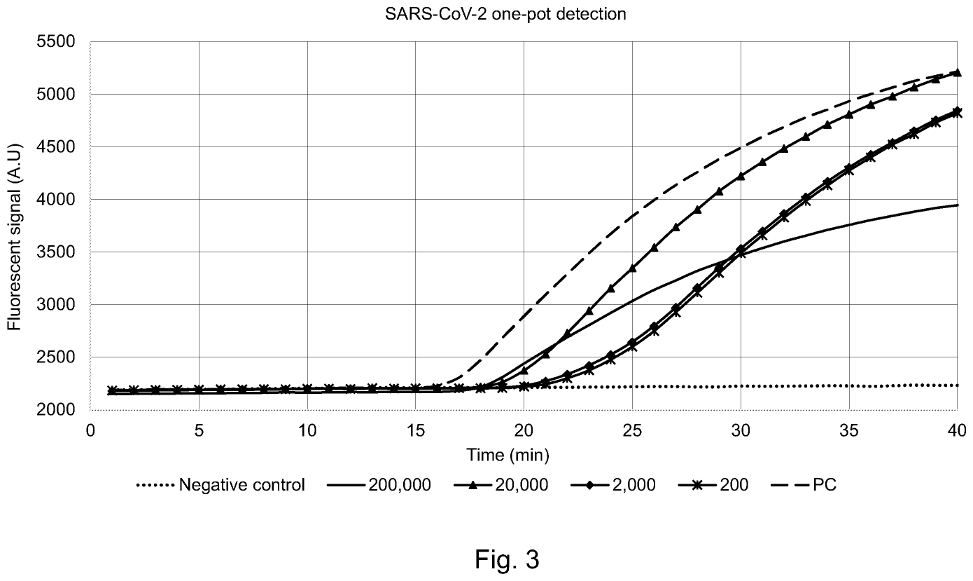

FIG. 4. In vitro transcription reaction using single stranded (ss) DNA templates A and B. “+” denotes presence, while “−” denotes absence of phiFa protein.

FIG. 5. In vitro PhiFa-44 RNA transcription assay, using 5′ fluorescent labelled (Cy3) single stranded DNA (50 nucleotides) as a template. One reaction was subjected to RNase H treatment. All products were analyzed in a native 4-20% (w/v) polyacrylamide gel. A fluorescent gel scanning was used to selectively visualize nucleic acids. Cy3 filter only shows the Cy3-labelled reaction products (left), whereas SYBR Gold staining and subsequent visualization with Cy2 filter shows all nucleic acids (right).

### 4 DETAILED DESCRIPTION OF THE INVENTION

### 4.1 Definitions

The term “phiFa_44”, as is used herein, refers to a protein having SEQ ID NO: 1. Said protein originates from the bacteriophage phiFa (GenBank: MH673672.2; Taxonomy ID: 1400796), which was isolated around mount Etna on Sicily, Italy (Severinov et al., 2014. Bacteriophage 4: e29399, DOI: 10.4161/bact.29399).

The term “RNA polymerase”, as is used herein in relation to phiFa_44, refers to a DNA dependent RNA polymerase that catalyzes the synthesis of a complementary strand of RNA from a DNA template.

The term “promoter”, as is used herein, refers to a nucleotide sequence at the 5′ end of a gene onto which the transcription initiation machinery, including an RNA polymerase such as a DNA-dependent RNA polymerase, binds and initiates transcription, Said promoter is often located 5-100 bp upstream of a start codon of gene.

The term “Clustered Regularly Interspaced Short Palindromic Repeats (CRISPR)”, as is used herein, refers to one or more specialized regions of DNA in the genome of prokaryotic microorganisms. These regions are characterized by the presence of nucleotide repeats that are interspersed by spacer sequences, typically direct DNA repeats of ˜25- to ˜38 bp, separated by unique spacer sequences of a similar length (Grissa et al., 2007. BMC Bioinformatics 8:172), which are derived from previous encounters with invading elements. They serve as a memory to quickly attack these invaders upon a next infection. The genomic region includes one or more genes encoding CRISPR-associated (Cas)-effector proteins that are located in the vicinity of the CRISPR loci.

The term “CRISPR RNA or crRNA”, as is used herein, refers to a CRISPR-derived RNA molecule comprising a spacer sequence and at least a 5′ repeat-derived terminus. Said crRNA preferably has a length of at least 30 nucleotides, more preferred at least 34 nucleotides, more preferred at least 40 nucleotides, more preferred at least 46 nucleotides. Said crRNA preferably is less than 1000 nucleotides, preferably less than 200 nucleotides, preferably less than 100 nucleotides. Said crRNA molecule may include ribonucleic acid nucleotide analogues such as inosine, uridine, xanthine, hypoxanthine, 2,6-diaminopurine, and 6,8-diaminopurine-based ribonucleotides and deoxyribonucleotides.

The term “CRISPR-associated (Cas) effector protein”, as is used herein, refers to a protein that is associated with crRNA. CRISPR/Cas systems are presently grouped into two classes. Class 1 systems utilize multi-subunit Cas complexes, whereas Class 2 systems use a single Cas protein to mediate its activity. Class 1, Type III CRISPR-Cas systems have evolved to target especially RNA sequences. Unique proteins in these systems are Cas3 in Class 1, Type I systems, Cas9 in Class 2, Type II systems, Cas10 in Class 1, Type III systems, Cas12 in class 2, Type V systems and Cas13 in Class 2, Type VI systems.

The term “effector complex”, as is used herein, refers to a CRISPR-Cas ribonucleoprotein complex that has (ribo) nuclease activity and may cleave and inactivate an invading nucleic acid sequence that comprises complementary sequences to the spacer sequence in the crRNA. Said complex comprises at least one crRNA and at least one Cas effector protein.

The term “reverse transcribing”, as is used herein, refers to the generation of complementary DNA (cDNA) from an RNA template. Retroviruses and some retrotransposons encode enzymes, termed reverse transcriptase, to replicate their genomes. A retroviral reverse transcriptase has three sequential biochemical activities: RNA-dependent DNA polymerase activity, ribonuclease H (RNAse H), and DNA-dependent DNA polymerase activity. Enzymes that are often used for reverse transcribing RNA are Moloney murine leukemia virus reverse transcriptase and avian myeloblastosis virus reverse transcriptase, and variants thereof, including thermostable variants.

The term “isothermal amplification”, as is used herein, refers to exponential amplification of nucleic acid molecules without thermal cycling, as is required for polymerase chain reaction (PCR). A polymerase with strand-displacement activity is usually employed in isothermal methods. Preferred single tube, isothermal reaction include nucleic acid sequence-based amplification (NASBA), loop-mediated isothermal amplification (LAMP), helicase-dependent amplification (HDA), recombinase polymerase amplification (RPA) reaction, and nicking enzyme amplification reaction (NEAR). A preferred single tube, isothermal reaction is a loop-mediated isothermal amplification (LAMP).

The term “Type V effector protein”, as is used herein, refers to a Class 2 effector protein that is characterized by a specific nuclease domain required for cleavage if a non-target desoxyribonucleic acid molecule. There are at least thirteen subtypes known to date: V-A, V-B, through V-M. Type V Cas proteins can be isolated from organisms such as Francisella novicida, Acidaminococcus sp., Lachnospiraceae sp., Prevotella sp., and some archeaebacteria. The total guide length preferably is 42-44 nucleotides. A gRNA for a Cas 12 Type V effector protein preferably comprises a dinucleotide TT as Protospacer Adjacent Motif (PAM) in the gRNA, more preferably a 5″-TTTN motif. Exemplary Type V effector proteins are Cpf1 (type V-A) and C2c1 (type V-B). Cas12 and Cas14 are prototype Type V effector proteins.

The term “Type VI effector protein”, as is used herein, refers to a Class 2 effector protein that has a nonspecific RNase activity. There are 4 subtypes known to date: subtypes VI-A, VI-B, VI-C, and VI-D. Type VI Cas proteins can be isolated from organisms such as Leptotrichia buccalis, Leptotrichia shahii, Ruminococcus flarefaciens, Bergeyella zoohelcum, Prevotella buccae, and Listeria seeligeri. The total guide length preferably is 52-66 nucleotides. A PAM motif for a Type VI effector protein is variable, as is known to a person skilled in the art, and may comprise a 3′ non-G for LshCas13a, a 5′ non-C and 3′ NAN or NNA (BzCas13b), or none (RfCas13d). Cas13 is a prototype Type VI effector protein.

The term “Type III Cas protein”, as is used herein, refers to a RNA-targeting, multiple subunit CRISPR-associated complex that comprises at least a Cas10 protein.

The term “Type IIIA Cas protein”, as is used herein, refers to an RNA-targeting Type III CRISPR/Cas protein complex that has unspecific DNase activity upon binding to a target RNA molecule. Type IIIA Cas protein includes, for example, Type IIIA Csm protein complexes from Staphylococcus thermophilus, Thermus thermophilus and Staphylococcus epidermis.

The term “Type III-B Cas protein”, as is used herein, refers to a RNA-targeting Type III CRISPR-Cas protein complex that also have unspecific DNase activity, with the exception of Type III-B Cas proteins from Thermus species. Said Type III-B Cas complex is composed of six to seven individual proteins. Type III-B Cas protein includes, for example, Type III-B Cmr protein complexes from Pyrococcus furiosus, Thermus thermophilus and Sulfolobus solfataricus.

The terms “quenched, quencher, and quenching”, as are used herein, refer to a process by which the signal intensity, preferably fluorescence intensity, of a given substance is decreased. Fluorescence quenching is a physicochemical process that absorbs emitted light from fluorescent molecules. Fluorescence quenching can be used as an indicator in nucleic acid diagnostics, where fluorophore and quencher molecules are attached to the ends of single-strand nucleic acid molecule and close to one another. As the nucleic acid molecule hybridizes to its target, or is cut by a nuclease, the fluorophore-quencher complex is pulled apart, allowing the fluorophore to produce light.

The term “dark quencher”, as is used herein, refers to a quencher that absorbs excitation energy from a fluorophore and dissipates the energy as heat. Hence, a dark quencher does not emit light itself. Dark quenchers are used in molecular biology in conjunction with fluorescent molecules.

The term “cyclic oligoadenylate (cOA)”, as is used herein, refers to a ring structure comprising 3-6 molecules of Adenosine Mono Phosphate (AMP). The formation of cOA is catalysed by the cyclase domain of Cas 10, which is part of Type III CRISPR/Cas effector systems.

The term “PPi” or pyrophosphate, as is used herein, refers to a salt or ester of pyrophosphoric acid. Alternative names are diphosphate and dipolyphosphate.

The term “inorganic pyrophosphatase”, or inorganic diphosphatase, as is used herein, refers to an enzyme that catalyzes the conversion of one ion of pyrophosphate to two phosphate ions. The enzyme is of the enzyme class EC 3.6.1.1.

The term “cOA-dependent effector protein”, as is used herein, refers to a protein of which the activity is dependent on the amount of cOA. Examples are ribonucleases such as endoribonucleases which degrade RNA non-specifically using a HEPN (Higher Eukaryotes and Prokaryotes, Nucleotide binding) active site. These nucleases get activated by binding of one or more cOA molecules using their CRISPR-associated Rossmann fold (CARF) domain. Examples of such non-specific effector endoribonucleases are the Cas accessory proteins Csx1 of Pyrococcus furiosus and Csm6 of Mycobacterium tuberculosis.

The term “non-naturally occurring protein”, as is used herein, refers to a protein that has an amino acid sequence and/or a post-translational modification pattern that is different to the protein in its natural state. For example, a non-naturally occurring protein may have one or more amino acid substitutions, deletions or insertions at the N-terminus, the C-terminus and/or between the N- and C-termini of the protein. A “non-naturally occurring” protein may have an amino acid sequence that differs from a naturally occurring amino acid sequence but that that is at least 80%, at least 85%, at least 90%, at least 95%, at least 97%, at least 98% or at least 99% identical to a naturally occurring amino acid sequence. In certain cases, a non-naturally occurring protein may contain an N-terminal methionine or may lack one or more post-translational modifications (e.g., glycosylation, phosphorylation, etc.) if it is produced by a different (e.g., bacterial) cell. In addition, a non-naturally occurring protein is tagged protein, comprising one or more specific tags by genetic engineering. Said tags include, but are not limited to, a c-myc domain, hemagglutinin tag, maltose-binding protein, glutathione-S-transferase, FLAG tag peptide, biotin acceptor peptide, streptavidin-binding peptide and calmodulin-binding peptide, as for example presented in Chatterjee, 2006 (Chatterjee, 2006. Cur Opin Biotech 17:353-358).

The term “non-naturally occurring nucleic acid”, as is used herein, refers to a nucleic acid that contains (1) a sequence of nucleotides that differs from a nucleic acid in its natural state; (2) one or more non-naturally occurring nucleotide monomers; and/or (3) one or more other modifications such as an added label or other moiety.

The term “non-naturally occurring composition” refers to a composition comprising (1) a combination of components that are not combined by nature, e.g., because they are at different locations, in different cells or different cell compartments; (2) a combination of components that have relative concentrations that are not found in nature; (3) a combination of components that lacks something that is usually associated with one of the components in nature; (4) a combination of components that is in a form that not found in nature, e.g., dried, freeze dried, crystalline, aqueous; and/or (5) a combination that contains a component that is not present in nature. For example, a preparation may contain a buffering agent, a detergent, a dye, a solvent or a preservative that does not naturally occur. A composition may be in any form, e.g., aqueous or lyophilized, and may be at any state, e.g. frozen or in an aqueous form.

The term “primer”, as is used herein, refers to an oligonucleotide, preferably of 15-50 nucleotides in length, that is effective in annealing to a template nucleic acid molecule and priming transcription, including reverse transcription, or replication of said template nucleic acid molecule by a polymerase. Said oligonucleotide may comprise deoxyribonucleotides, ribonucleotides, or a combination or variants thereof. Said variants include synthetic oligonucleotide analogues such as phosphorothioate, phosphotriester, phosphorothioate 2-alkylated, and phosphoramidate analogues, analogues with modifications at the 2′-position of nucleoside sugar rings such as 2′-fluoro, O-methyl, or methoxyethyl, peptide nucleic acid, bridged nucleic acid, and/or locked nucleic acid molecules.

### 4.2 PhiFa_44 Protein

4.2.1 At present, no DNA dependent RNA polymerase that remains active for more than 5 minutes at 65° C. is commercially available. In search of a thermostable DNA dependent RNA polymerase, bacteriophages that may infect thermophilic bacteria such as Thermus thermophilus were analyzed. The bacteriophage phiFa (GenBank: MH673672.2; Taxonomy ID: 1400796) was identified as a candidate phage. PhiFa is an as yet unclassified Oshimavirus belonging to the Siphoviridae phages with long non-contractile tails. PhiFa encodes a hypothetical protein “phiFa_44” that was predicted to be an “RNA polymerase” (GenBank: QKE11339.1; SEQ ID NO:1).

BlastP searches identified a hypothetical protein from Escherichia coli, WP_164703602.1, as having a relatively high identity match of 56.79% over about 90% of the length of the phiFa_44 protein. However, upon closer inspection WP_164703602.1 appears to be a contaminant in the sequencing analysis, as an alignment was not possible to the E. coli genome. In addition, the encoding sequence is uploaded to the database as a separate contig. The true origin of WP_164703602.1 is not known. No homologies were found between phFa_44 and bacteriophage-derived RNA polymerases SP6, T3 and T7.

One further aligning sequence was a hypothetical protein from Planctomycetes bacterium (MBI5851759.1), which scored about 30% over 56% of the length of the phiFa_44 protein. A final aligning sequence was again a hypothetical protein from E. coli (WP_206306715.1), which scored about 47% over only 10% of the length of the phiFa_44 protein.

Searches in the Pfam database (Mistry et al., 2021. Nucleic Acids Res 49: D412-D419) with the PhiFa_44 amino acid sequence identified only one result over a length of 257 amino acid residues with a score of only 0.019. This score is not significant. The one result that was identified (RdRP; PF05183) belongs to a family of eukaryotic RNA dependent RNA polymerases. These proteins are involved in post transcriptional gene silencing where they are thought to amplify dsRNA templates.

Searches employing the HHpred tool in the Teubingen toolkit (available at toolkit.tuebingen.mpg.de/) identified two sequences, 5FSW_C and 2J7N_B, with very low sequence similarity (25.8% and 17.5%, respectively). Both proteins that were identified are predicted to be RNA dependent RNA polymerases. Sequences with even less sequence similarity (<8%) were identified as multi-subunit DNA dependent RNA polymerases.

Both types of insignificant scores, RNA-dependent RNA polymerase and multi-subunit RNA polymerase, are not considered likely in the context of phiFa_44.

An in silico structural analysis was performed to gain more insight into the phiFa_44 protein. Initial results showed some similarity to QDE1 RNA-dependent RNA polymerases, which similarity was at best 22% sequence identity over a region of only 207 amino acid residues. However, this domain resembles a magnesium (Mg) binding domain. Predicted 3D structures (data not shown) confirmed these similarities and point to amino residues D366, D368 and D370 as the residues in phiFa_44 that may be responsible for Mg binding.

In view of the absence of significant sequence similarities with known RNA polymerases such as bacteriophage-derived RNA polymerases SP6, T3 and T7, it was decided not to pursue phiFa_44 as a potential heat-stable RNA polymerase. However, one final IVT experiment that was performed (see Example 1) suggested that, despite the low sequence identity, phiFa_44 might function as a heat-stable RNA polymerase.

4.2.2 A protein having at least 50% sequence identity to SEQ ID NO:1 according to the invention may be expressed and purified from a suitable expression system.

Commonly used expression systems for heterologous protein production include E. coli, Bacillus spp., baculovirus, yeast, fungi, filamentous fungi or yeasts such as Saccharomyces cerevisiae and Pichia pastoris, eukaryotic cells such as Chinese Hamster Ovary cells (CHO), human embryonic kidney (HEK) cells and PER.C6® cells (Thermo Fisher Scientific, MA, USA), and plants. The efficiency of expression of a recombinant protein in a heterologous system depends on many factors, both on the transcriptional level and the translational level.

A protein having at least 50% sequence identity to SEQ ID NO:1 according to the invention preferably is produced in a prokaryotic cell, preferably E. coli. Said protein is preferably produced by expression cloning of the protein in a prokaryotic cell of interest, preferably E. coli. Said expression construct, preferably DNA, is preferably produced by recombinant technologies, including the use of polymerases, restriction enzymes, and ligases, as is known to a skilled person. Alternatively, said expression construct is provided by artificial gene synthesis, for example by synthesis of partially or completely overlapping oligonucleotides, or by a combination of organic chemistry and recombinant technologies, as is known to the skilled person.

As an alternative, or in addition, a protein having at least 50% sequence identity to SEQ ID NO:1 according to the invention may be isolated from a thermophilic organism by expression of a tagged protein having at least 50% sequence identity to SEQ ID NO:1 according to the invention in said thermophilic organism, and isolation of said protein on the basis of the tag.

Said expression construct is preferably codon-optimised to enhance expression of the protein having at least 50% sequence identity to SEQ ID NO:1 according to the invention in a prokaryotic cell of interest, preferably E. coli. Further optimization may include removal of cryptic splice sites, removal of cryptic polyA tails and/or removal of sequences that lead to unfavorable folding of the mRNA. In addition, the expression construct preferably encodes a protein export signal for secretion of the protein having at least 50% sequence identity to SEQ ID NO: 1 according to the invention out of the cell into the periplasm of a prokaryote, allowing efficient purification of the protein having at least 50% sequence identity to SEQ ID NO:1 according to the invention.

Methods for purification of a protein having at least 50% sequence identity to SEQ ID NO:1 according to the invention are known in the art and are generally based on chromatography such as affinity chromatography and ion exchange chromatography, to remove contaminants. In addition to contaminants, it may also be necessary to remove undesirable derivatives of the product itself such as degradation products and aggregates. Suitable purification process steps are provided in Berthold and Walter, 1994 (Berthold and Walter, 1994. Biologicals 22:135-150).

As an alternative, or in addition, a recombinant protein having at least 50% sequence identity to SEQ ID NO:1 according to the invention may be tagged with one or more specific tags by genetic engineering to allow the protein to attach to a column specific to the tag and therefore be isolated from impurities. The purified protein is then exchanged from the affinity column with a decoupling reagent. The method has been increasingly applied for purifying recombinant protein. Conventional tags for proteins, such as histidine tag, may be used with an affinity column that specifically captures the tag (e.g., a Ni-IDA column for a histidine tag) to isolate the protein from other impurities. The protein is then exchanged from the column using a decoupling reagent according to the specific tag (e.g., imidazole for histidine tag). This method is more specific, when compared with traditional purification methods.

Suitable further tags include c-myc domain, hemagglutinin tag, maltose-binding protein, glutathione-S-transferase, FLAG tag peptide, biotin acceptor peptide, streptavidin-binding peptide and calmodulin-binding peptide, as presented in Chatterjee, 2006 (Chatterjee, 2006. Cur Opin Biotech 17, 353-358). Methods for employing these tags are known in the art and may be used for purifying a protein having at least 50% sequence identity to SEQ ID NO:1 according to the invention.

Methods for expression proteins in E. coli are known in the art and can be used for expression and purification of a protein having at least 50% sequence identity to SEQ ID NO:1 according to the invention.

### 4.3 Methods of Transcribing a Nucleic Acid Template

The invention provides a thermostable, DNA dependent RNA polymerase that allows in vitro transcription reactions to be carried out at elevated temperatures without debilitating loss of catalytic activities. Said elevated temperature may increase specific activity and may help to alleviate secondary structures from the resulting RNA molecule. In addition, the availability of a thermostable RNA polymerase allows a one-pot reaction for a nucleic acid detection strategy involving amplification at an elevated temperature, such as LAMP amplification, and synthesis of RNA strands, especially detection systems involving Type III CRISPR-Cas systems.

Said thermostable RNA polymerase according to the invention has at least 50% sequence identity to SEQ ID NO:1, preferably at least 60% sequence identity, preferably at least 70% sequence identity, preferably at least 80% sequence identity, preferably at least 90% sequence identity, preferably at least 91% sequence identity, preferably at least 92% sequence identity, preferably at least 93% sequence identity, preferably at least 94% sequence identity, preferably at least 95% sequence identity, preferably at least 96% sequence identity, preferably at least 97% sequence identity, preferably at least 98% sequence identity, preferably at least 99% sequence identity, preferably at least 99.5% sequence identity, with SEQ ID NO:1. At least 99.5% sequence identity with SEQ ID NO:1 means that one amino acid residue may differ between said thermostable RNA polymerase and SEQ ID NO:1.

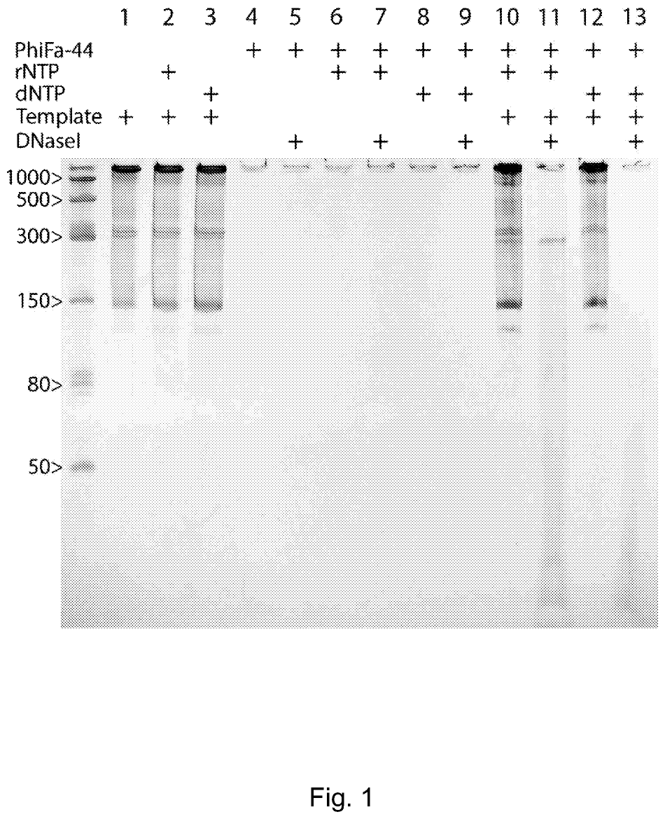

A suitable template nucleic acid molecule is or comprises deoxynucleic acid nucleotides, and comprises a single stranded (ss) DNA molecule, a double stranded (ds) DNA molecule, or a hybrid ss-ds molecule. It was found that a thermostable RNA polymerase according to the invention may generally transcribe either single stranded or double stranded DNA molecules into RNA molecules. Said general, low transcriptional activity apparently does not require a specific promoter region. As is shown in FIG. 1, initiation of transcription of a ds DNA template by a thermostable RNA polymerase such as phiFA_44 appears to occur randomly, resulting in a ladder of RNA products.

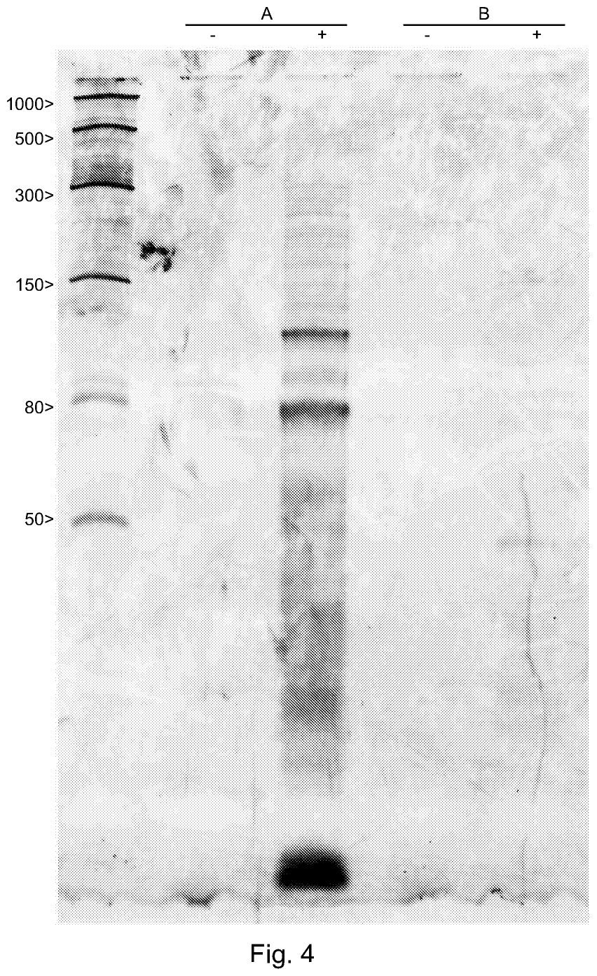

It was further found that a thermostable RNA polymerase according to the invention such as phiFA_44 specifically transcribe especially single stranded DNA molecules into RNA molecules, whereby the template strand comprises a consensus sequence GGGGCGG, preferably AGGGGCGG, more preferably TAGGGGCGG, more preferably TAGGGGCGGM, more preferably TAGGGGCGGMTA, which may function as a phiFa_44 promoter sequence, whereby M denotes either a C or an A. A sequence CAGGGGCGGCAA, comprising the consensus sequence GGGGCGG, is present in the ssDNA template A that was used for the IVT reaction shown in FIG. 4. Said sequence CAGGGGGGCAA may have functioned as a promoter sequence, resulting in an RNA transcript of about 70 nucleotides, as is shown in FIG. 4. A related sequence may have driven transcription from a discrete transcription start site resulting in a more defined RNA product of about 300 nt in FIG. 1.

An in vitro transcription reaction involving a thermostable RNA polymerase according to the invention is performed in a suitable buffering agent to keep the pH at a nearly constant value. Said buffering agent may include phosphate, borate, N-cyclohexyl-2-aminoethanesulfonic acid (CHES), tris(hydroxymethyl)aminomethane (Tris), 2-(N-morpholino) ethanesulfonic acid (MES), glycine, and/or [tris(hydroxymethyl)methylamino]propanesulfonic acid (TAPS). A preferred reaction pH for an RNA polymerase is between 6 and 10, preferably between 7 and 9, such as 7.2, 7.5, 7.8, 8.0, 8.1, 8.5. 8.6 or 8.8.

A preferred buffering agent for a transcription reaction is or comprises Tris, preferably 10-100 mM Tris. Said Tris preferably is set at a desired pH by the addition of an acid such as acetate and/or hydrogen chloride.

A suitable buffer for transcription reactions may further comprise one or more divalent metal ions such as a Mg²⁺ or Mn²⁺. Divalent metal ions may be provided as salts thereof such as magnesium chloride, magnesium sulphate and/or magnesium acetate. Said concentration preferably is between 0.5 and 20 mM, such as between 1 and 10 mM, preferably about 2 mM, about 3 mM, about 5 mM, or about 7 mM.

Additional components of said buffer may include potassium ions, such as potassium chloride, other salts such as ammonium sulphate, and/or betaine, ethylene glycol, 1,2-propanediol and/or spermidine, as known in the art to enhance transcription of a template nucleic acid molecule, such as 2-10% DMSO or 2-10% glycerol (Cheng et al., 1994. Proc Natl Acad Sci 91:5695-5699).

Preferably, additional ingredients are included in the reaction buffer to stabilize enzyme activity including gelatin, albumin, an reducing agent such as one or more of beta-mercaptoethanol, dithiothreitol (DTT), and/or tris(2-carboxyethyl) phosphine (TCEP), and/or a mild detergent such as, for example, TWEEN 20 or Triton-X 100.

Said transcription reaction is carried out in the presence of ribonucleotides (NTP), comprising a nucleobase and a ribose sugar group that is coupled to a triphosphate group. Said nucleobase includes adenine, guanine, cytosine, uracil, and any modifications thereof. The term ribonucleotide includes reference to an analogue of a ribonucleotide such as a fluorescent molecule, for example 1,3-diaza-2-oxophenothiazine-ribose-5′-triphosphate (tCTP), and/or other analogues such as inosine, xanthosine, N4-hydroxycytosine, N4-methoxycytosine and 6H, 8H-3,4-dihydropyrimido[4,5-c][1,2]oxazin-7-one (Suzuki et al., 2005. Nucleic Acids Symp Series 49:97-98).

Said transcription reaction is carried out at a temperature between 30° C. and 80° C., preferably between 45° C. and 75° C., such as between 50° C. and 70° C., including 55° C., 60° C. and 65° C.

Said transcription reaction is preferably carried out in the presence of 10-50 mM, preferably 20 mM, Tris-HCl pH 8.8, 100-1000 nanoM (nM), preferably about 500 nM of a thermostable RNA polymerase according to the invention, 1-5 mM, preferably about 2.5 mM, NTP mix (NEB #N0466S), 1-10 mM, preferably about 2 mM, MgCl, 10-250 mM, preferably about 50 mM, NaCl and a DNA template, preferably 1-100 ng such as about 25 ng, of dsDNA template in a total volume of 20 microL at a temperature of 45° C.-75° C., such as between 50° C. and 70° C., including 55° C., 60° C. and 65° C.

Said IVT reaction is performed for a suitable amount of time to transcribe the DNA template, preferably said double stranded DNA template. Said amount of time preferably is 0.01-1 hour, more preferably 0.1-0.5 hour, more preferred 0.2-0.4 hour.

### 4.4 Diagnostic Assays

4.4.1 The methods of the invention allow detecting specific nucleic acid sequences which may be used especially in human healthcare and veterinary diagnostics. For such purposes, nucleic acid material, including DNA and/or RNA, is preferably isolated from a sample. For this, said nucleic acid material may be purified using, for instance, a combination of physical and chemical methods. Commercially available systems for nucleic acid isolation are preferably used, such as the NucliSENS® easyMAG® or NucliSENS® miniMAG® nucleic acid extraction system (bioMérieux, Marcy l'Etoile, France), or a MagNA Pure 96 System (Roche Diagnostics, Almere, The Netherlands).

Said sample may include a biological fluid, such as saliva, an upper respiratory specimen such as a nasopharyngeal swab, a lower respiratory specimen such as sputum, nasopharyngeal secretion, oropharyngeal secretion, sweat, urine, stool, or blood. The term “blood” includes blood plasma, which is prepared by removing red and white blood cells, for example by centrifugation, and blood serum, which is prepared by formation of a blood clot, and removal of the clot using, for example, a centrifuge. Methods and compositions for isolation of nucleic acid material from biological fluids, particularly swabs, preferably employ aqueous solvents without use of organic solvents and chaotropic salts.

For this, RNA may be isolated from a sample by any technique known in the art, including but not limited to suitable commercial RNA isolation kits such as Trizol (Invitrogen; Carlsbad, California), RNAqueous® (Applied Biosystems/Ambion, Austin, Tx), Qiazol® (Qiagen, Venlo, The Netherlands), Agilent Total RNA Isolation Lits (Agilent; Santa Clara, California), RNA-Bee® (Tel-Test. Friendswood, Texas), the RNeasy mini kit (Qiagen, Venlo, The Netherlands), and Maxwell™ 16 Total RNA Purification Kit (Promega; Madison, Wisconsin).

The isolated RNA is preferably reverse transcribed with the aid of a RNA-dependent DNA polymerase into single or double stranded complementary DNA (cDNA), using methods known to a person skilled in the art. Reverse transcription may be primed by universal primers, such as random hexamers or nonamers, or by one or more specific primers such as virus-specific or even gene specific primers.

DNA, including genomic DNA, may be isolated from a sample by any technique known in the art, including but not limited to suitable commercial DNA isolation kits such as Quick-DNA Viral Kits (Zymo Research; Irvine, CA), NucleoSpin Dx Virus (Macherey-Nagel, Düren, Germany), QIAamp DNA Blood Mini Kit (Qiagen, Venlo, The Netherlands), and EasyPure® Viral DNA/RNA Kit (Transgen Biotech, Beijing, China).

4.4.2 The resulting RNA, DNA or cDNA may be used directly in a diagnostic CRISPR/Cas effector protein-based assay according to the invention but, preferably is amplified prior to detection to increase detection levels. Amplification may be performed by any suitable amplification system including, for example, ligase chain reaction (LCR), isothermal ribonucleic acid amplification systems such as nucleic acid sequence-based amplification (NASBA), cleavage-based signal amplification of RNA (Zhao et al., 2013. Nature Comm 4:1493), transcription mediated amplification, strand displacement amplification and, polymerase chain reaction (PCR).

A preferred amplification reaction is a single tube, isothermal reaction such as NASBA, loop-mediated isothermal amplification (LAMP), helicase-dependent amplification (HDA), rolling circle amplification (RCA), multiple displacement amplification (MDA), recombinase polymerase amplification (RPA) reaction, and nicking enzyme amplification reaction (NEAR). A preferred single tube, isothermal reaction is a loop-mediated isothermal amplification (LAMP) or a recombinase polymerase amplification (RPA) reaction.

LAMP normally employs six primers that recognize distinct target sequences on the template strand. Four of these primers are “inner primers” (LF, LB, FIP and BIP) which are designed to synthesize new DNA strands. The outer primers (F3 and B3) anneal to the template strand and also generate new DNA. These primers are accompanied by a DNA polymerase which aids in strand displacement and releases the newly formed DNA strands. This amplification takes place in less than one hour such as in less than 20 minutes, under isothermal conditions between 60-65° C.

The RPA process (TwistDx Ltd., Cambridge, UK) employs a recombinase, a single-stranded DNA-binding protein (SSB) and strand-displacing polymerase, preferably supplemented with a reverse transcriptase, more preferably with a reverse transcriptase and a DNA-dependent RNA polymerase such as a T7 polymerase. This amplification takes place in less than one hour, under isothermal conditions at 37-42° C.

4.4.3 A thermostable RNA polymerase according to the present invention may replace a DNA-dependent RNA polymerase in an amplification reaction such as NASBA, cleavage-based signal amplification of RNA (Zhao et al., 2013. Nature Comm 4:1493), transcription mediated amplification, or RPA, thereby allowing a higher temperature for said amplification reaction.

### 4.5 CRISPR/Cas-Based Detection Systems

4.5.1 A thermostable RNA polymerase according to the invention may further be used in clustered regularly interspaced short palindromic repeats (CRISPR) nucleic acid detection system, comprising a CRISPR-associated effector protein (Cas) and at least one CRISPR RNA (crRNA) that binds to a target nucleic acid molecule.

Said CRISPR/Cas-based detection system preferably is provided as a sensing device comprising a CRISPR-based nucleic acid system according to the invention, or “biosensor”. A signal that is generated when the CRISPR-based nucleic acid system interacts with a nucleic acid molecule that is complementary to the crRNA, for example a colorimetric, fluorometric, fluorescent or bioluminescent signal, may be coupled to a transducer, allowing quantification of the signal. The signal may be directly visualized or, as an alternative or in addition, be converted by means of a suitable transducer into a measurable electrical parameter such as a current or voltage.

4.5.2 Binding of the crRNA to a target sequence results in activation of the Cas effector protein. Activation of said Cas effector protein may be detected, for example, by collateral cleavage of a reporter molecule as is mediated by, for example, by Cas12, Cas13 and Cas14, and/or the generation of cyclic oligoadenylate (cOA) which is, for example, catalysed by the cyclase domain of Cas10.

Crispr/Cas Class 1 systems utilize multi-protein effector complexes (Koonin et al., 2017. Curr Opin Microbiol 37:67-78) and include Type I systems, Type III such as Cas10) and Type IV systems. Crispr/Cas Class 2 systems utilize single-protein effectors and include Type II CRISPR systems such as Cas9, Type V systems such as Cas12 (also known as cpf1) and Cas 14 (Harrington et al., 2019, Science 362:839-842), and Type VI systems such as Cas13 (Makarova et al., 2017. Cell 168:328-328).

4.5.3 Binding of a crRNA to its target nucleic acid sequence activates a collateral, trans-acting nuclease activity of Type Vor Type VI CRISPR/Cas effector protein. This collateral, trans-acting nuclease activity allows the use of a single-stranded “reporter” molecule to score activation of a Type V or Type VI CRISPR/Cas effector protein.

A reporter for an activated Type Vor Type VI Cas preferably is a single stranded nucleic acid molecule of at least 9 nucleotides, cleavage of which by the activated Type V or Type VI Cas can be detected. Said single stranded nucleic acid molecule preferably comprises 9-50 nucleotides, more preferably 10-25 nucleotides or even 11-20 nucleotides, such as 12 nucleotides, 13 nucleotides, 14 nucleotides, 15 nucleotides, 16 nucleotides, 17 nucleotides, 18 nucleotides, or 19 nucleotides. An optimal length of said reporter is about 12 nucleotides.

Said reporter either comprises single stranded DNA for detection of an activated Cas12 or Cas14 effector protein, or single stranded RNA for detection of an activated Cas13 effector protein. Said reporter may include deoxyribonucleotide or ribonucleotide analogues such as inosine, uridine, xanthine, hypoxanthine, 2,6-diaminopurine, and 6,8-diaminopurine-based ribonucleotides and deoxyribonucleotides.

Detection of reporter cleavage may be performed by any method known in the art. For example, detection may be performed directly by mass spectrometry, for example ultra-high performance liquid chromatography (UHPLC) coupled to tandem mass spectrometry (LC-MS/MS) in positive electrospray ionization mode. The LC-MS/MS analysis may be performed, for example by using a high end UHPLC chromatographic system coupled to a triple-quadrupole mass-spectrometer. Detection may further be performed by liquid-liquid phase separation (LLPS; Spoelstra et al., 2018. BioRXiv, CSHL (doi.org/10.1101/471482), or by a colorimetric, fluorometric, fluorescent or bioluminescent detection method that is known to a person skilled in the art.

An appropriate reporter is a single stranded RNA (Cas13) or DNA (Cas12 and Cas14) molecule that is tagged with a fluorescent label on one end and a quencher on the other end. The close proximity of the reporter to the quencher prevents detection of its fluorescence. Cleavage of the substrate after activation of a collateral, trans-acting nuclease activity of Type V or Type VI CRISPR/Cas effector protein breaks the reporter-quencher proximity and thus allows unquenched emission of fluorescence, which can be detected after excitation with a laser.

A preferred fluorescent label may be selected from Atto425 (ATTO-TEC GmbH, Siegen, Germany), Atto 647N (ATTO-TEC GmbH, Siegen, Germany), YakimaYellow (Epoch Biosciences Inc, Bothell, WA, USA), Cal610 (BioSearch Technologies, Petaluma, CA, USA), Cal635 (BioSearch Technologies, Petaluma, CA, USA), FAM (Thermo Fisher Scientific Inc., Waltham, MA USA), TET (Thermo Fisher Scientific Inc., Waltham, MA USA), HEX ((Thermo Fisher Scientific Inc., Waltham, MA USA), cyanine dyes such as Cy5, Cy5.5, Cy3, Cy3.5, Cy7 (Thermo Fisher Scientific Inc., Waltham, MA USA), Alexa dyes (Thermo Fisher Scientific Inc., Waltham, MA USA), Tamra (Thermo Fisher Scientific Inc., Waltham, MA USA), ROX (Thermo Fisher Scientific Inc., Waltham, MA USA), JOE (Thermo Fisher Scientific Inc., Waltham, MA USA), fluorescein isothiocyanate (FITC, Thermo Fisher Scientific Inc., Waltham, MA USA), Yakima Yellow® (YY; Epoch Biosciences, Bothell, Washington) and tetramethylrhodamine (TRITC, Thermo Fisher Scientific Inc., Waltham, MA USA). Said substrate is preferably labeled at the 5′ end with a detectable label, preferably a fluorescent label.

Quenchers, for example tetramethylrhodamine TAMRA, dihydrocyclopyrroloindole tripeptide minor groove binder, are known in the art. Preferred quenchers include Black Hole Quencher®-1 (BHQ1) and BHQ2 (Biosearch Technologies, Petaluma, CA, USA). The BHQ1 dark quencher has strong absorption from 480 nm to 580 nm, which provides quenching of fluorophores that fluoresce in this range, such as FAM, TET, CAL Fluor® Gold 540, JOE, HEX, CAL Fluor Orange 560, and Quasar® 570 dyes. The BHQ2 dark quencher has strong absorption from 599 nm to 670 nm, which provides quenching of fluorophores that fluoresce in this range, such as Quasar® 570, TAMRA, CAL Fluor® Red 590, CAL Fluor Red 610, ROX, CAL Fluor Red 635, Pulsar® 650, Quasar 670 and Quasar 705 dyes. BHQ1 and BHQ2 may quench fluorescence by both FRET and static quenching mechanisms.

A further preferred reporter allows detection in a lateral flow assay (LFA). The principle of LFA is that a reaction product, in the present case an unreacted reporter is immobilized at a first point, termed control line, while the cleaved reporter is immobilized at a second point, termed test line. LFAs are typically composed of a nitrocellulose membrane, sample pad, conjugate pad, wicking or absorbent pad and backing pad (Jauset-Rubio et al., 2016. Sci Rep 6:37732). Nitrocellulose membranes are most commonly used as they facilitate a support capable of use for both reaction and detection, with capture biomolecules e.g. antibodies, are deposited on the nitrocellulose to form the test and control lines via a combination of electrostatic interactions, hydrogen bonds and/or hydrophobic interactions (Jauset-Rubio et al., 2016. Sci Rep 6:37732).

4.5.4 As an alternative, binding of a crRNA to its target nucleic acid sequence activates a cyclase domain of Type III effector systems such as Cas 10. A Cas10-based ribonucleic acid detection system preferably is derived from a thermophilic organism such as Pyrococcus furiosus, Sulfolobus solfataricus or Thermus thermophilus. For this system, a target DNA molecule needs to be transcribed into a target RNA molecule, for example by a thermostable RNA polymerase according to the invention, thereby allowing a higher temperature for said transcription reaction. A further advantage is that all three steps, amplification, transcription and detection can be employed at an elevated temperature, for example at about 65° C., rendering all steps suitable for a one-pot reaction.

A further advantage of a CRISPR/Cas-based ribonucleic acid detection system from a thermophilic organism is that detection can be performed at an elevated temperature, for example between 40° C. and 80° C., preferably between 50° C. and 70° C., such as between 55° C. and 65° C., preferably about 65° C. Incubation at this temperature may accelerate the cOA synthesis reaction, when compared to incubation at a lower temperature. In addition, said elevated temperature may inactivate a nuclease, such as a DNase or RNase, or protease that is present in the sample.

In addition, an advantage of a CRISPR/Cas-based ribonucleic acid detection system from a thermophilic organism may provide an increased stability to the system such as it may be stored for a longer period of time, when compared to a CRISPR/Cas-based ribonucleic acid detection system from a mesophilic organism.

Resent results indicate that the presence of cOA leads to a large increase in RNase activity by Csm6 or Csx1 family members. These proteins are often encoded in the Type III loci, but do not associate directly with the ribonucleoprotein complex. Instead, recognition of invader RNA transcripts may result in targeted RNA degradation by Cas7, non-specific DNA degradation by a Class I, Type IIIA Cas10, as well as cOA production leading to the activation of Csm6 or Csx1, thereby causing collateral cleavage of other nearby single stranded RNA molecules.

4.5.5 Methods of directly determining a level of cOA preferably include methods to determine a level of pyrophosphate or PPi. The formation of pyrophosphate is coupled to the formation of cOA from ATP by a CRISPR/Cas associated protein such as Cas10. The formation of cOA, consisting of 3-6 AMP units, results in the simultaneous formation of 3-6 molecules of PPi.

4.5.6 As an alternative, the detection system according to the invention may comprise an inorganic pyrophosphatase, resulting in the breakdown of PPi and the formation of two inorganic phosphate molecules for each PPi molecule. Hence, by determining a level of inorganic phosphate, the number of detectable molecules is amplified from 1 molecule of cOA to 6-12 molecules of Pi.

A preferred inorganic pyrophosphatase is an enzyme that is active at the same or similar temperature as the Type III CRISPR/Cas-based RNA detection system. In addition, it is preferred that the inorganic pyrophosphatase is active under the same or similar conditions as the Type III CRISPR/Cas-based RNA detection system, including at the same or similar pH and at the same or similar salt concentrations. For example, if the Type III CRISPR/Cas-based RNA detection system has an optimal activity at 50° C., it is preferred that the inorganic pyrophosphatase is active at this temperature. Preferably, the activity of the inorganic pyrophosphatase at 50° C. is such that essentially all PPi molecules are broken down into inorganic phosphate molecules. Said break down preferably is instantaneous. Similarly, the activity of the inorganic pyrophosphatase at the chosen pH and salt concentrations is such that essentially all PPi molecules are broken down into inorganic phosphate molecules. Said break down preferably is instantaneous.

A preferred inorganic pyrophosphatase is from a thermophylic organism such as Pyrococcus furiosus, Sulfolobus solfataricus and Thermus thermophilus, allowing simultaneous, isothermal detection. In this way, a level of cOA can be directly determined by determining a level of Pi.

4.5.7 PPi and Pi can be detected using methods known in the art, including colorimetric-, fluorometric-, fluorescent- or bioluminescent-based assays.

Suitable methods for determining a level of PPi include a fluorometric and/or colorimetric pyrophosphate (PPi) Assay Kit (Biovision Inc., Milpitas, CA); a fluorescent EnzChek® Pyrophosphate Detection Kit (ThermoFisher Scientific; Waltham, MA); a fluorometric Pyrophosphate Assay Kit (SigmaAldrich, Saint Louis, MO); and a luminescent PPiLight™ assay (Lonza Group A.G., Bazel, Switzerland).

Suitable methods for determining a level of inorganic phosphate or Pi include a colorimetric PiColorLock™ assay (Expedeon, Cambridge, UK); a colorimetric Malachite Green Phosphate Assay Kit (SigmaAldrich, Saint Louis, MO); a fluorescent Phosphate Sensor (ThermoFisher Scientific; Waltham, MA); a luminescent readout following conversion of ADP to ATP (US patent application US20140273036A); a fluorescent chemosensor (Meng et al., 2015. RSC Advances 5:53189-53197); and photoluminescent graphene quantum dots combined with Europium ions (Bai et al., 2013. Chemistry 19:3822-3826).

The methods and means for directly determining a level of cyclic oligoadenylate (cOA) preferably include at least one substrate and, if required, an enzyme that allows at least one of the indicated detection methods for PPi or Pi.

A preferred method is a colorimetric method such as the Malachite Green Phosphate Assay Kit, allowing fast determination of a level of PPi or Pi, as a direct determination of a level of cOA.

4.5.8 Methods of indirectly determining a level of cOA preferably include methods to determine an activity of a cOA-dependent, non-specific effector nuclease such as CRISPR ancillary nuclease 1 (Can1) or Can2 and, preferably, methods to determine an activity of cOA-dependent, non-specific effector endoribonuclease such as Csx1. For this, a detection system according to any the invention preferably includes a cOA-dependent, non-specific effector endoribonuclease such as Csx1.

Said cOA-dependent, non-specific effector nuclease, preferably endoribonuclease, preferably is an enzyme that is active at the same or similar temperature as the Type III CRISPR/Cas-based RNA detection system. In addition, it is preferred that the cOA-dependent, non-specific effector endoribonuclease is active under the same or similar conditions as the Type III CRISPR/Cas-based RNA detection system, including at the same or similar pH and at the same or similar salt concentrations. For example, if the Type III CRISPR/Cas-based RNA detection system has an optimal activity at 65° C., it is preferred that the cOA-dependent, non-specific effector endoribonuclease is active at this temperature. Preferably, the activity of the cOA-dependent, non-specific effector endoribonuclease at 65° C. is such that the cOA-induced activity of the cOA-dependent, non-specific effector endoribonuclease results in the production of detectable amounts of a reaction product of the substrate of said cOA-dependent, non-specific effector endoribonuclease. Similarly, the activity of the cOA-dependent, non-specific effector endoribonuclease at the chosen pH and salt concentrations is such that cOA-induced activity of the cOA-dependent, non-specific effector endoribonuclease results in the production of detectable amounts of a reaction product of the substrate of said cOA-dependent, non-specific effector endoribonuclease.

A substrate for the cOA-dependent, non-specific effector endoribonuclease preferably is a RNA molecule, cleavage of which can be detected. Detection may be performed by any method known in the art. For example, detection may be performed directly by mass spectrometry, for example ultra-high performance liquid chromatography (UHPLC) coupled to tandem mass spectrometry (LC-MS/MS) in positive electrospray ionization mode. The LC-MS/MS analysis may be performed, for example by using a high end UHPLC chromatographic system coupled to a triple-quadrupole mass-spectrometer.

Detection may further be performed by liquid-liquid phase separation (LLPS; Spoelstra et al., 2018. BioRXiv, CSHL (doi.org/10.1101/471482).

A preferred substrate for the cOA-dependent, non-specific effector endoribonuclease is a RNA molecule that is tagged with a fluorescent reporter molecule on one end and a quencher on the other end. The close proximity of the reporter to the quencher prevents detection of its fluorescence. Cleavage of the substrate by activation of the cOA-dependent, non-specific effector endoribonuclease breaks the reporter-quencher proximity and thus allows unquenched emission of fluorescence, which can be detected after excitation with a laser. An increase in the activity of the cOA-dependent, non-specific effector endoribonuclease therefore causes a proportional increase in fluorescence due to the cleavage of the substrate and removal of the quencher that quenches the fluorescent reporter.

Separate from activation of the non-specific effector endoribonuclease, or in addition thereto, determination of a level of target recognition may also be performed by determination of activation of the non-specific effector DNase activity that is present in Type IIIA CRISPR/Cas effector complexes using an appropriate substrate for said nuclease. Said appropriate substrate preferably is a DNA molecule that is tagged with a fluorescent label on one end and a quencher on the other end. Examples of suitable fluorescent labels and quenchers are provided herein above. The close proximity of the reporter to the quencher prevents detection of its fluorescence. Cleavage of the substrate by activation of the cOA-dependent, non-specific effector DNase breaks the reporter-quencher proximity and thus allows unquenched emission of fluorescence, which can be detected after excitation with a laser. An increase in the activity of the cOA-dependent, non-specific effector DNase therefore causes a proportional increase in fluorescence due to the cleavage of the substrate and removal of the quencher that quenches the fluorescent reporter. The use of both the activation of the non-specific effector endoribonuclease and the activation of the non-specific effector DNase allows determination of presence or absence of two independent target RNA molecules in one single assay, provided that two independent ribonucleoprotein complexes are used, one of which specifically activates the non-specific effector DNase while the other specifically allows indirect or direct determination of cOA levels. A person skilled in the art will understand that for this, the fluorescent labels that are present on substrate by activation of the cOA-dependent, non-specific effector endoribonuclease and the non-specific effector DNase must be sufficiently different to allow determination of a level of each activity as a measure for determining a level of cOA.

A suitable commercial substrate is provided by the RNaseAlert® Lab Test Kit v2 (ThermoFisher Scientific; Waltham, MA).

A preferred cOA-dependent, non-specific effector endoribonuclease is from a thermophylic organism such as Pyrococcus furiosus, Sulfolobus solfataricus and Thermus thermophilus, allowing simultaneous, isothermal detection of the activity of the cOA-dependent, non-specific effector endoribonuclease.

The methods and means for indirectly determining a level of cyclic oligoadenylate (cOA) preferably include a cOA-dependent, non-specific effector endoribonuclease and a substrate of said cOA-dependent, non-specific effector endoribonuclease.

For the purpose of clarity and a concise description, features are described herein as part of the same or separate embodiments, however, it will be appreciated that the scope of the invention may include embodiments having combinations of all or some of the features described.

### 5 EXAMPLES

### Example 1

### Materials & Methods

### Protein Purification

The protein of interest nucleotide sequence was codon optimized for E. coli (see Table 1) and placed into a bicistronic expression plasmid (Nieuwkoop et al., 2019. Microbiol Biotechnol 12:173-179) containing a Strep-tag at the N-terminus. The expression plasmid was transformed into E. coli strain BI21 (DE3) and grown at 37° C. in a desired culture volume (2-12 L) until an OD600 of about 0.6 was reached. The culture was then placed on ice for 1 hour, after which IPTG was 10 added to a final concentration of 0.5 mM. The culture was then incubated at 20° C. for about 16 hours (overnight). The cells were harvested and lysed in Buffer A (100 mM Tris-HCl, 150 mM NaCl, pH 8) by sonication and subsequently spun down at 30.000 g for 45 min. The clarified lysate was filtered (0.45 microM) and purified using a StrepTrap FPLC column. The protein of interest was eluted using Buffer B (100 mM Tris-HCl, 150 mM NaCl and 2.5 mM desthiobitin, pH 8.0). Subsequently, a Heparin purification step was performed using Buffer C (100 mM Tris-HCl, 125 mM NaCl, pH 8) as binding and washing buffer. Subsequent gradient elution was performed using Buffer D (100 mM Tris-HCl, 1 M NaCl, pH 8). Finally, size exclusion chromatography was performed using a Superdex 200 increase 10/300 GL column (Cytiva; Marlborough, MA) in Buffer A.

### IVT Assay

The IVT assays were performed using a base composition of 1× isothermal amplification buffer (NEB #B0537S), 250 nM phiFa_44, 1.875 mM rNTP/dNTP, 80 ng dsDNA template in a total volume of 20 microL. These reactions were incubated at 65° C. for 1 h with a subsequent incubation at 95° C. for 10 min. Thereafter, 2.3 microL of DNasel Buffer and 1 microL of DNasel enzyme (NEB #B0303, #M0303, respectively) were added and the reactions were incubated at 37° C. for 30 min. The reactions mixtures were resolved on a 10% UREA-PAGE gel and stained with SybrGold (Thermo #S11494).

### Temperature and NaCl Concentration Optimum Analysis

The IVT assays were performed using a base composition of 20 mM Tris-HCl pH 8.8, 500 nM phiFa_44, 2.5 mM rNTP mix (NEB #N0466S), 2 mM MgCl, 50 mM NaCl and 25 ng of dsDNA template in a total volume of 20 microL. To determine an optimal NaCl concentration, the reaction mix was identical with only the NaCl concentration varying. The reactions were incubated at 65° C. for 1 h after which the reaction were cleaned up with the Zymo RNA clean & concentrator kit (Zymo #R1017). Subsequent DNase treatment was performed for 1 h at 37° C. in a total reaction volume of 20 microL (NEB #M0303S). The reactions mixtures were resolved on a 10% UREA-PAGE gel and stained with SybrGold (Thermo #S11494).

### ScopeDx Assay

The ScopeDx assay mixture consists of 1× Warmstart LAMP mastermix (NEB #E1700), primers (FIP/BIP 1.6 microM, F3/B3 0.2 microM, LoopF/LoopB 0.4 microM), 62.5 nM reconstituted TtCmr46 complex with E-gene crRNA (Steens et al., 2021. Nature Comm 12:1-12), 1 microM TtHB144 (Steens et al., 2021. Nature Comm 12:1-12), 250 nM rNTP mix (NEB #N0466), 500 nM RNaseAlert V2 (Thermo #4479769) and 175 nM phiFa_44. Various amounts of the Twist SARS-COV-2 synthetic genome MN908947.3 were added to the reaction mixtures and incubated at 65° C., while time-point measurements were taken on FAM-channel.

### Results

### In Vitro Transcription Assay

To determine whether dsDNA is a suitable template, a stretch from the phiFa phage genome was ordered as a synthetic gene. A region was chosen that contains the end of a phiFa-45 an intergenic region and the start of the phiFa_44 gene (see Table 1). The in vitro transcription assay was performed as is indicated in FIG. 1, designed to determine whether the polymerase uses rNTPs or dNTPs as a substrate and whether DNA or RNA is generated.

From these results, we concluded that the template utilized is dsDNA, which is used along with rNTP substrates to generate a RNA product (see lanes 10-13 of FIG. 1).

### Temperature and NaCl Concentration Optimum Analysis

The IVT assays performed using various NaCl concentrations and temperatures show that optimal NaCl concentration is 25-200 mM and optimal temperature range is 45-75° C.

### One-pot ScopeDx

In the ScopeDx 1.0 tool, as described in Steens et al., 2021. Nature Comm 12:1-12, we were able to link the LAMP pre-amplification (65° C.) with the CRISPR read-out (65° C.). However, due to the incompatibility of the used commercial Hi-T7 RNA polymerase with extended incubation at 65° C., we had to add a step in the protocol. First, the LAMP pre-amplification was performed at 65° C. for ˜30 min, then the Hi-T7+CRISPR mixture was added to the reaction and incubated for an additional 10-20 min at 65° C. In this way the Hi-T7 RNA polymerase, with its limited duration of activity, was able to transcribe the dsDNA created by the pre-amplification into RNA which can then be detected by the CRISPR system.

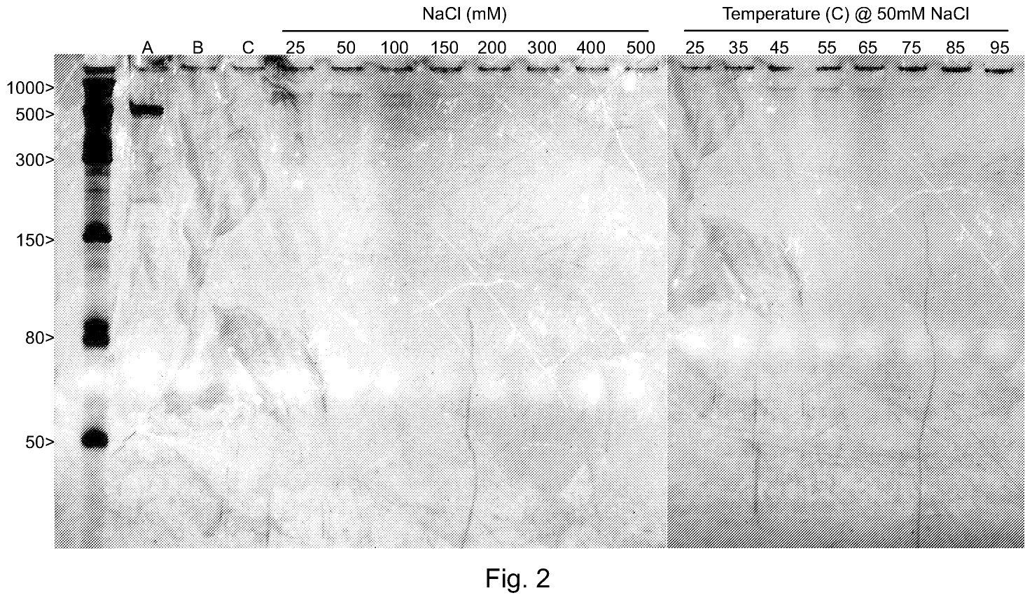

Since phiFa_44 is able to transcribe DNA into RNA at 65° C. we decided to test this in a one-pot 1-step assay, ScopeDx 2.0. The assay was performed on a synthetic SARS-COV-2 RNA genome as a single step at 65° C. (FIG. 2). With this new assay, a limit of detection of 200 copies was reached in less than 25 minutes, showing a huge improvement in time-to-signal. Furthermore, the fact that it is now a 1-step protocol greatly reduces contamination risks, which can occur if it is required to open the tube in between steps, and improves high throughput and automation capabilities.

### In Silico Structural Analysis

An in silico structural analysis was performed to get more insight into the phiFa_44 protein. Initial results show similarity to QDE1 polymerase-like enzymes. The dimerizing behavior of QDE1 could resemble how phiFa_44 acts as well. For now, based on the amino acid alignment (see FIG. 4) the magnesium binding domain could be identified. Predicted 3D structures confirm these similarities and point to D366, D368 and D370 as the residues in phiFa_44 responsible for Mg binding.

### Example 2

### Materials & Methods

### IVT Assay

In vitro transcription (IVT) assays were performed using a base composition of 1× isothermal amplification buffer (NEB #B0537S), 250 nanoM PhiFa-44, 1.875 mM rNTP/dNTP, 80 ng double stranded (ds) DNA template or 100 nM (final concentration) single stranded (ss) DNA oligo in a total volume of 20 microL. These reactions were incubated at 65° C. for 1 h with a subsequent incubation at 95° C. for 10 min. A total of 2.3 and 1 microL, respectively, were added of DNasel Buffer and DNasel enzyme (NEB #B0303, #M0303), and the reactions were incubated at 37° C. for 30 min. The reactions mixtures were resolved on a 10% UREA-PAGE gel and stained with SybrGold (Thermo #S11494).

The ssDNA template A was 5′-GCGCTCCAAAAGCAGGGGCGGC
AAGTCCAGTCTCCAGACTTGCCGCCGAGTGTACCTGCTTGCTACAGCCCT
AATTATACCACAAACGCC 3′.
The ssDNA template B was 5′-GGCGTTTGTGGTATAATTAGGG
CTGTAGCAAGCAGGTACACTCGGCGGCAAGTCTGGAGACTGGACTTGCCG
CCCCTGCTTTTGGAGCGC 3′.

The dsDNA template was composed of annealed ssDNA template A and ssDNA template B.

### Results

PhiFa IVT ssDNA Templates

An IVT reaction was performed using ssDNA templates of 90 nt in length, template B being complementary to template A (see FIG. 4). This region of the PhiFa genome was chosen because it contained a potential motif of interest. From the results it can clearly be seen that ssDNA template A resulted in a clear ssRNA transcript of about 75-80 nt, while ssDNA template B did not provide a clear transcript. This results indicates that ssDNA template A contains a motif of interest that is preferred by the PhiFa44 polymerase.

Very little is known about how phage PhiFA expresses its genes. Typically, phages use host RNA polymerases to express them, but since phage PhiFA encodes for its own RNA polymerase, it is reasonable to assume that (at least) some of its genes are expressed by this particular polymerase (encoded by the PhiFA_44 gene).

### Example 3

### Materials and Methods

### In Vitro RNA Transcription Assay

PhiFa-44 was purified (His) 6-tagged in 50 mM NaCl, 100 Mm Tris-HCl pH 8.0, and a single-stranded DNA template (50 nt; SEQ ID NO:12) was ordered 5′ labelled with Cy3 fluorescent dye. The ssDNA template was purified from a denaturing PAGE using the “ZR Small-RNA PAGE Recovery kit” (ZymoResearch, R1070). In vitro RNA transcription assays were conducted in PhiFa-44 reaction buffer (35 mM NaCl, 20 mM Tris-HCl pH 8.8, 2 mM MgCl2, and 25 mM rNTPs), and supplemented with Milli-Q to a final volume of 20 μl. When required, the ssDNA template and PhiFa-44 were added to the reaction mix to a final concentration of 62 \dot{η}M and 1 μM, respectively.

All reactions were incubated at 65° C. for 2 h, followed by an RNA/DNA clean up using the “RNA clean & Concentrator kit” (ZymoResearch, R1017). After adding RNase H (NEB, 5 U/μl) to the selected purified sample, all reactions were incubated at 37° C. for 30 min. To analyze the reaction products, 2×RNA loading dye (200 mM Tris-HCl pH 8.0, 30% glycerol, 900 mM NaCl) was added to all incubation samples, after which they were loaded on a native 4-20% (w/v) gradient polyacrylamide gel. The gel was visualized using fluorescent gel scanning (GE Amersham Typhoon). The Cy3 filter (560-580 nm) was used to visualize Cy3-labelled reaction products, whereas staining with SYBR Gold and applying the Cy2 filter (515-535 nm) showed all nucleic acids present on the gel.

### Results

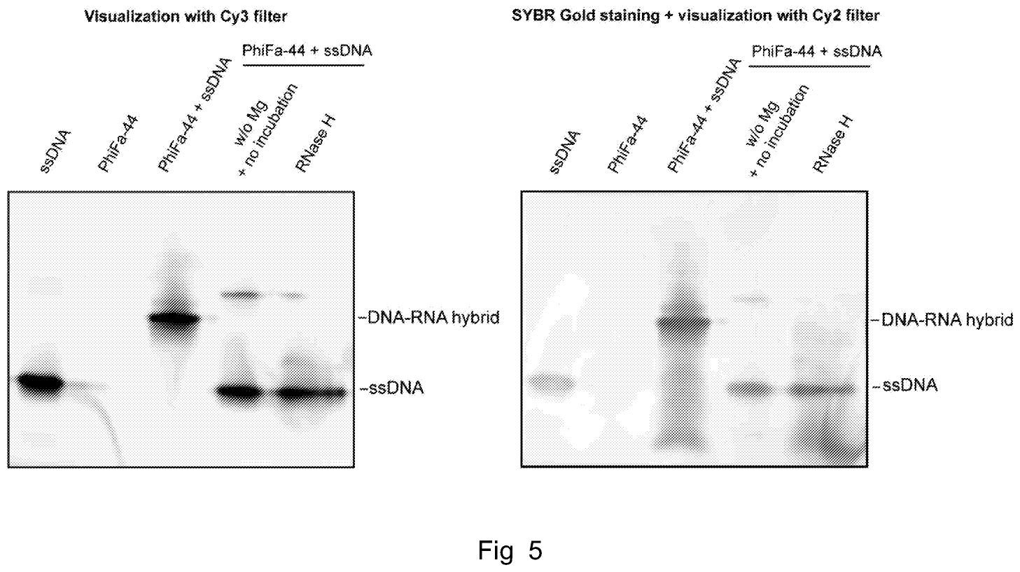

As is shown in FIG. 5, in vitro transcription using PhiFa-44 and ssDNA as template results in the formation of complementary RNA, indicated by the shifting of the ssDNA template band. When MgCl was not added and no incubation was performed (negative control), no shifting was observed. Similarly, addition of RNase H, an enzyme cleaving RNA in a RNA:DNA duplex, resulted in abrogation of the shifting of the ssDNA template band.

These results show that PhiFa-44 is able to transcribe single stranded template DNA into RNA.

| SEQ | | |
|---|---|---|
| ID NO | Name | Sequence |
| 1 | QKE11339.1 | MNKTELVKKLIEEKTAELAVRPVYTGRIDEEGQWTPTSLHERYQDK |
| | [Thermusphage | VLVVPAPEEGEPVQTIGEYEFVGGNSNRIVYIHRSIIKQWLEVAGG |
| | phiFa] | DSHWGAAVVLRPGKLASHNAVESWEGYQLEESDEGLLATMDERDWK |
| | | VLLEAYDLDPEVRLVQVTVEDLEQGVSAKGTLRKVLPGERPVVEPN |
| | | SWKKFGNLRTQFMSILTTDVHYNPRASVNLQELTYYVQEAPAQWVE |
| | | DALDEEIERIINLKTSPWLTTGYLTRLGFSPAWNSTLTALKESLCG |
| | | ELKKLLRRIKLSGKYGLRAKSTSSHLLRDGELLLPAYAKKHVKVGD |
| | | FVTVSRNPALPSQGWAKYRVTGFIDGNMVVFPQGDYAWSAILGGDH |
| | | DGDDAVVFYRAPVEGYELPDDTLDLQAIKPVARKLDANTVEARIQR |
| | | WQNEVSVNIGQFDLAARRLLAANKLHREEAKLLSVAIQTAISLKKR |
| | | VARLEDQSWWPRVAELLEESKKLAGTTWVDMIRDGREPVGAPDWVL |
| | | TIYQKVHEAIKKVQKLEPRLWLTEAKVREFAKGATVPASCEALIQY |
| | | REKLLKAKAIAVQNEDGDTVARLNKELKEFIHVEAPKHLLGIDEED |
| | | WKQFSRWAIGNTHISEWVEWCHPAILEELYDRFGKQTIKAIALGGT |
| | | PNLAESEVIELEDQELIDRVFEVEGVKYALLDETGYLKAGRLKVTK |
| | | VRKSVVEFEYA |
| 2 | E. coli | ATGAACAAAACCGAGTTAGTTAAGAAACTCATAGAGGAAAAAACTG |
| | harmonized | CAGAACTCGCTGTACGTCCTGTTTATACCGGGAGGATCGATGAGGA |
| | Phifa_ | GGGGCAGTGGACTCCTACTTCGCTGCACGAACGTTATCAGGATAAG |
| | 44 | GTTCTGGTAGTACCTGCACCTGAAGAAGGGGAGCCGGTTCAGACCA |
| | | TCGGTGAGTATGAGTTTGTTGGGGGGAACTCGAACAGGATCGTTTA |
| | | TATCCATCGTTCGATCATCAAACAATGGCTGGAGGTTGCAGGGGGT |
| | | GACAGCCACTGGGGGGCAGCAGTAGTACTGCGTCCGGGTAAACTCG |
| | | CTAGCCATAACGCAGTTTTTTCGTGGGAGGGTTATCAGTTAGAGGA |
| | | ATCGGATGAGGGTCTGCTGGCAACTATGGACGAACGTGATTGGAAA |
| | | GTACTGCTGGAGGCTTATGATCTGGATCCTGAAGTACGTCTCGTTC |
| | | AGGTTACTGTTTTCGATCTGGAGCAGGGTGTTAGCGCAAAAGGTAC |
| | | TCTGCGTAAAGTTCTCCCTGGGGAGCGTCCTGTAGTTTTTCCTAAC |
| | | TCGTGGAAAAAATTTGGTAACCTCCGTACTCAATTTATGAGCATCC |
| | | TGACCACTGACGTTCACTATAACCCCCGTGCTAGCGTTAACCTACA |
| | | AGAGCTCACCTATTATGTTCAGGAGGCTCCCGCTCAGTGGGTTTTT |
| | | GACGCTCTGGATGAAGAGATCGAACGTATCATAAACCTGAAAACCA |
| | | GCCCTTGGCTGACTACTGGTTATCTGACCCGTCTCGGTTTCAGCCC |
| | | TGCTTGGAACTCGACCCTCACCGCTCTCAAGGAAAGCCTGTGCGGT |
| | | GAACTGAAGAAACTACTGCGTCGTATAAAACTGTCGGGTAAATATG |
| | | GTCTGCGTGCAAAATCGACTTCGAGCCACCTCCTGCGTGATGGTGA |
| | | ATTACTGTTACCTGCATACGCAAAAAAACACGTTAAGGTTGGTGAT |
| | | TTCGTTACTGTTTCGCGTAACCCTGCACTACCTTCGCAGGGGTGGG |
| | | CAAAATATCGTGTAACTGGGTTTATAGATGGTAACATGGTAGTTTT |
| | | TCCTCAGGGGGATTATGCATGGAGCGCTATCCTGGGTGGTGATCAT |
| | | GATGGTGACGACGCAGTTGTATTTTATCGTGCTCCGGTTGAAGGTT |
| | | ATGAACTCCCGGACGACACTCTGGATCTGCAGGCAATAAAACCCGT |
| | | AGCACGTAAACTCGACGCAAACACCGTAGAGGCAAGGATACAACGT |
| | | TGGCAAAACGAGGTAAGCGTTAACATCGGTCAGTTCGATCTGGCTG |
| | | CTAGGCGTCTCCTAGCAGCAAACAAGCTGCATCGTGAGGAGGCAAA |
| | | ACTACTGTCGGTAGCAATACAGACTGCAATCAGCCTCAAAAAACGT |
| | | GTAGCTCGTCTAGAAGATCAGAGCTGGTGGCCGCGTGTAGCTGAGC |
| | | TGCTCGAAGAGAGCAAAAAACTCGCTGGTACCACTTGGGTTGATAT |
| | | GATACGTGATGGTCGTGAGCCGGTAGGGGCACCCGACTGGGTTCTC |
| | | ACCATCTATCAGAAAGTTCATGAAGCAATCAAGAAAGTTCAGAAGC |
| | | TGGAACCCCGTCTGTGGCTCACTGAAGCAAAAGTACGTGAATTCGC |
| | | AAAGGGGGCAACTGTACCTGCAAGCTGCGAGGCTCTAATCCAGTAT |
| | | CGTGAAAAACTCCTGAAAGCTAAAGCAATCGCAGTTCAGAACGAGG |
| | | ATGGTGATACTGTTGCACGTTTAAACAAAGAACTGAAGGAGTTTAT |
| | | CCACGTAGAGGCACCGAAACATCTGCTGGGGATAGATGAAGAAGAT |
| | | TGGAAACAGTTTAGCAGGTGGGCAATCGGGAACACCCATATCAGCG |
| | | AATGGGTTTTTTGGTGCCACCCCGCTATCCTAGAAGAACTGTATGA |
| | | TCGTTTCGGGAAACAAACCATCAAAGCTATCGCACTGGGTGGGACT |
| | | CCGAACCTAGCAGAAAGCGAGGTAATCGAGCTGGAAGATCAGGAAC |
| | | TAATAGATCGTGTATTTGAAGTTGAAGGTGTAAAATATGCTCTGTT |
| | | AGATGAAACCGGTTATCTCAAGGCAGGGCGTCTGAAAGTTACTAAG |
| | | GTTCGTAAATCGGTTGTAGAATTTGAATATGCTTGA |
| 3 | PhiFa | AATTCTAATACGACTCACTATAGtatccggtgggccagccagcgta |
| | phage | ccgtcctcgaggctgtggattgcgcggcttcccgcccgcagccgcg |
| | template | catctagtcctcccgccaccaccacctctgcaggaagaggtgggcg |
| | | aagagacgaagagaggtgaaagagagaagaggcgaaggccgggtgt |
| | | cccgcgcccggctcgccttacggtgccacgccaccccgctcgcgca |
| | | aggctccgcgcggagcgcggccccttcgggcttacggccttgcgct |
| | | cgcggggaccccggcgtggcagggctccgccccgagccgggcgcgt |
| | | agaggacacccggctcaaggaggtcaaagatgaacaagaccgaact |
| | | tgtcaaaaagttgattgaagagaagacggcggagttggctgttagg |
| | | ccggtgtacaccggtcgaatcgacgaagaaggtcagtggactccga |
| | | cttctctg |
| 4 | Template | ctgtggacgctgacttcatcgacgtactggaagatgtcaaaaattg |
| | | aggcgtttgtggtataattagggctgtagcaagcaggtacactcgg |
| | | cggcaagtctggagactggacttgccgcccctgcttttggagcgcc |
| | | gggtggtttgggcgcggctttatgtttcgcccatggggggagccac |
| | | ccggctttatcctcccccgactggaggctagatgcagcttcgcaag |
| | | attgcgccgaacgccaacttcttccgcctccttgaggagaacttca |
| | | gcgcggaactggaagacatggacgctctgcccatctaggaggttgg |
| | | atgaacatccgagatgcccccaagctgttcctcggagctttcgggc |
| | | tttcgaccccagctacgtggctgcggtggagagcatcgcgaagcgg |
| | | aagctctgagaggtgaagcatggcgaggttcactggatttagcaag |
| | | gtcttctcggtcggcaagacgctggagaaccaccgcatcaagttct |
| | | tcagctacgctaccgtctagaccgcgtaagggtggggagggggcag |
| | | tctctccccacccacctgttacctggaggtgaacgtgcgagtgcgt |
| | | ttgcgtaaggcggtgtacgctagcggcaaactccatgcggcgggga |
| | | gtgttcaaggtgatttctggccttgtgttcttccgcgtgtgaggtg |
| | | agagatggcgatttttgagctgaacgctggttccgaccttcgcttc |
| | | cccaccaggaagga |
| 5 | LAMP | AGCTGATGAGTACGAACTT |
| | E-gene | |
| | F3 | |
| 6 | LAMP | TTCAGATTTTTAACACGAGAGT |
| | E-gene | |
| | B3 | |
| 7 | LAMP | ACCACGAAAGCAAGAAAAAGAAGTATTCGTTTCGGAAGAGACAG |
| | E-gene | |
| | FIP | |
| 8 | LAMP | TTGCTAGTTACACTAGCCATCCTTAGGTTTTACAAGACTCACGT |
| | E-gene | |
| | BIP | |
| 9 | LAMP | CTGCGCTTCGATTGTGTGCGT |
| | E-gene | |
| | Loop | |
| 10 | LAMP | AGCTGATGAGTACGAACTT |
| | E-gene | |
| | F3 | |
| 11 | E-gene | AUUGCGACACAAUAUUGCAGCAGUACGCACACAAUCGAAGCGCAGA |
| | crRNA | |
| 12 | Template | 5′ Cy3-CGATGGCCGTAGATAAGGCGCCCGGGGACGACCACGTCA |
| | | AGGCGCAGTTC |

1. A method comprising
incubating a template nucleic acid molecule with a protein having at least 50% sequence identity with SEQ ID NO: 1 in the presence of ribonucleic acid nucleotides (rNTPs) and a suitable buffer; and
transcribing at least part of said template nucleic acid molecule into an RNA molecule by performing a transcription reaction at a temperature between 30° C. and 80° C.

2. The method of claim 1, wherein said template nucleic acid molecule is a deoxyribonucleic acid (DNA) template molecule.

3. The method of claim 1, wherein said buffer comprises 25-200 mM of NaCl.

4. The method of claim 1, wherein the transcription reaction is performed at a temperature between 45° C.-75° C.

5. The method of claim 1, wherein the template nucleic acid molecule is generated by a pre-amplification reaction.

6. The method of claim 1, wherein the transcribed RNA molecule is further incubated with a clustered regularly interspaced short palindromic repeats (CRISPR)-CRISPR-associated (Cas) protein based nucleic acid detection system.

7. The method of claim 6, wherein the CRISPR-Cas nucleic acid detection system comprises:
a) an effector complex comprising a Type III Cas protein and at least one CRISPR RNA (crRNA) that binds a target RNA molecule;
b) means for directly or indirectly determining a level of cyclic oligoadenylate (cOA).

8. The method of claim 6, wherein the pre-amplification reaction, transcription reaction and incubation with a nucleic acid detection system are all performed in a one pot reaction.

9. A protein having at least 50% sequence identity with SEQ ID NO:1.

10. The protein of claim 9, which comprises a suitable tag.

11. A nucleic acid molecule encoding the protein of claim 9.

12. The nucleic acid molecule of claim 11, which is codon optimized for expression of said protein in a suitable host cell.

13. A host cell expressing the protein of claim 9.

14. (canceled)

15. (canceled)
